(19) United States
(12) Patent Application Publication
(10) Pub. No.: US 2022/0186259 A1
(43) Pub. Date: Jun. 16, 2022

(54) CELL EXPANSION WITH SELF-REPLICATING RNA VECTORS EXPRESSING IMMORTALIZATION PROTEINS

(71) Applicant: EMD Millipore Corporation, Burlington, MA (US)

(72) Inventor: Naohisa Yoshioka, Wildomar, CA (US)

(21) Appl. No.: 17/605,190

(22) PCT Filed: Jun. 25, 2020
(86) PCT No.: PCT/US2020/039585

§ 371 Date: Oct. 20, 2021

Related U.S. Application Data
(60) Provisional application No. 62/867,505, filed on Jun. 27, 2019.

(51) Int. Cl. C12N 15/86; C12N 9/12

(52) U.S. Cl. CPC C12N 15/86; C12N 2770/36143; C12N 2510/04; C12N 9/1276

(57) ABSTRACT

Synthetic, self-replicating RNA vectors comprising sequence encoding at least one immortalization protein, and use of the self-replicating RNA vectors to extend the lifespan of primary cell populations and expand said populations of primary cells.

### RELATED APPLICATIONS

The present application claims the benefit of priority of U.S. Priority Patent Application No. 62/867,505, filed Jun. 27, 2019, the entire contents of which is incorporated herein in its entirety.

### FIELD

The present disclosure relates to compositions and methods for expanding populations of primary cells.

### BACKGROUND

Primary cells are isolated directly from human or animal tissue. As such, they are more similar to the in vivo state and exhibit normal cell physiology. Primary cells, however, are mortal and have a limited lifespan, i.e., they stop dividing (or senesce) after a certain number of cell divisions. Immortalization genes such as telomerase, cellular oncogenes, and viral oncogenes have been used for immortalization of primary cells. However, integration of these immortalization genes, e.g., viral oncogenes, into the genome of primary cells can alter the integrity of the primary cells. Means for reversibly immortalizing primary cells would be very beneficial.

### SUMMARY

Among the various aspects of the present disclosure is the provision of self-replicating RNA vectors encoding one or more immortalization proteins. In general, the self-replicating RNA vectors are based on an alphavirus (such as, e.g., Venezuelan equine encephalitis virus) in which sequence encoding viral structural proteins is deleted and replaced with sequence encoding at least one immortalization protein. In some embodiments, the at least one immortalization protein is chosen from human telomerase (hTert), human papillomavirus type 16 E6 protein (HPV16 E6), human papillomavirus type 16 E7 protein (HPV16 E7), simian vacuolating virus 40 large T antigen (SV40 LT), cMyc-T58A protein, homeobox HoxB8 protein, homeobox HoxA9 protein, homeobox HoxA10 protein, adenovirus E1A protein, adenovirus E1B protein, cyclin-dependent kinase 4 (CDK4), Ras V12 protein, polycomb complex protein Bmi1, hsp70 member 9 (HSPA9), or combination thereof.

Another aspect of the present disclosure provides primary cells comprising any of the self-replicating RNA vectors disclosed herein.

Still another aspect of the present disclosure encompasses plasmid vectors encoding any of the self-replicating RNA vectors disclosed herein.

A further aspect of the present disclosure provides methods for extending lifespan in populations of primary cells, the methods comprising introducing into a population of primary cells any of the self-replicating RNA vectors disclosed herein, wherein upon expression of the at least one immortalization protein, the population of primary cells has an increased lifespan as compared to a population of control primary cells not transfected with the self-replicating RNA vector and/or not exposed to the at least one immortalization protein.

Yet another aspect of the present disclosure encompasses methods for expanding populations of primary cells, the methods comprising (a) introducing into a population of primary cells any of the self-replicating RNA vector disclosed herein, wherein upon expression of the at least one immortalization protein, the population of primary cells has an increased lifespan as compared to a population of control primary cells not transfected with the self-replicating RNA vector and/or not exposed to the at least one immortalization protein; and (b) removing the self-replicating RNA vector from the population of primary cells by dilution and/or via an interferon innate immune response once the population of primary cells reaches an appropriate cell quantity.

Other aspects and iterations of the present disclosure are described in more detail below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 presents schemes showing the structure of several self-replications RNA vectors disclosed herein.

FIG. 2 shows images of mock transfected and RFP-hTert transfected BJ fibroblasts after the cells were subjected to a senescence associated beta-galactosidase staining protocol.

FIG. 3 presents fluorescent (left) and bright field (right) images of RFP-hTert (no E3L) transfected and RFP-hTert-E3L transfected MSCs.

FIG. 4 presents bright filed images of mock transfected, RFP-hTert-E3L transfected, RFP-hTert-E6E7-E3L transfected, and RFP-htert-T58A-E3L transfected adult HEK cells.

FIG. 5A shows bright filed images of RFP-hTert-E3L transfected, RFP-hTert-E6E7-E3L transfected, and RFP-htert-T58A-E3L transfected MSCs before and after removal of the self-replicating RNA vector.

FIG. 5B illustrates expression of MCS marker genes, CD44 and CD105, before and after removal of the indicated self-replicating RNA vectors.

FIG. 6 presents bright field (left) and fluorescence+bright field (right) images of HEK cells transfected with the indicated self-replicating RNA vectors.

FIG. 7A shows GFP expression of S-ATG, S-ATA, S-ATC, S-ATT, P-ATG, P-ATA, P-ATC, and P-ATT measured by flowcytometry. Cells were cultured in the presence of puromycin (P), B18R protein (B), and Ruxolitinib (R) for 4 weeks.

FIG. 7B presents fluorescent images the GFP expression of S-ATG, S-ATA, S-ATC, S-ATT, P-ATG, P-ATA, P-ATC, and P-ATT on Day 14.

FIG. 8A shows GFP expression of all 20 kinds of amino acids for the position at nsP2-773 with the ATC version on day 1 by flowcytometry in HFFs.

FIG. 8B shows GFP expression of 14 kinds of amino acids for the position at nsP2-773 with the ATC version on Day 7 by flowcytometry in HFFs.

FIG. 8C shows GFP expression of 14 kinds of amino acids for the position at nsP2-773 with the ATC version on Day 14 by flowcytometry in HFFs.

FIG. 8D shows GFP expression of all 20 kinds of amino acids for the position at nsP2-773 with the ATC version on Day 1 by flowcytometry in NIH3T3 cells.

FIG. 8E shows GFP expression of 8 kinds of amino acids for the position at nsP2-773 with the ATC version on Day 7 by flowcytometry in NIH3T3 cells.

FIG. 8F shows GFP expression of 8 kinds of amino acids for the position at nsP2-773 with the ATC version on Day 14 by flowcytometry in NIH3T3 cells.

FIG. 9A presents fluorescent images of GFP on Day 5 after transfection with the S-ATG-E6E7-GFP2 and the W-ATC-E6E7-GFP2 in HFFs.

FIG. 9B shows the population doubling level (PDL) after transfection with the S-ATG-E6E7-GFP2 and the W-ATC-E6E7-GFP2 into adult HEK cells. Cells were cultured in the presence of B18R protein (200 ng/mL), Puromycin (0.2-0.8 μg/mL), and Ruxolitinib (1 μM).

FIG. 10 shows the summary of mutations at the third nucleotide of VEE genome and the amino acid position at nsP2-773.

### DETAILED DESCRIPTION

The present disclosure provides synthetic self-replicating RNA vectors comprising sequence encoding at least one immortalization protein. The self-replicating RNA vectors can be used to provide transient immortalization to populations of primary cells and expansion of said cell populations. Moreover, the integrity of primary cells is maintained because there is no integration of immortalization sequences into the genome of the cells. Once the desired degree of cell expansion has been achieved, the self-replicating RNA vectors can be removed from the cells by dilution or via an interferon immunity response.

### (I) Self-Replicating RNA Vectors Encoding Immortalization Proteins

One aspect of the present disclosure provides synthetic self-replicating RNA vectors that encode at least one immortalization protein. The self-replicating RNA vectors are based on modified alphaviruses in which sequence encoding viral structural proteins has been deleted and replaced with sequence encoding at least one immortalization protein. The self-replicating RNA vectors comprise sequence encoding a plurality of replication complex proteins, which ensure replication of the RNA vector over several cell generations, but the viral vectors do not form infectious particles due to the deletion of the viral structural genes. Upon entry into a cell, the RNA vector serves as a template for translation of the viral replication complex proteins and the one or more immortalization proteins. The replicated RNA cannot recombine with cellular DNA, and thus, there is no risk of integrating immortalization genes into the genome of the cell. Moreover, in the absence of genomic integration, the integrity of the primary cells can be maintained.

### (a) Synthetic Self-Replicating RNA

The synthetic self-replicating RNA (or replicon) contains all the sequence elements needed for translation of the encoded proteins and replication of the RNA vector. In particular, the replicon is based on a modified alphavirus in which the non-structural replicase genes are maintained and the structural genes (needed to make an infectious particle) are removed. In various embodiments, the modified alphavirus can be derived from Aura virus, Babanki virus, Barmah Forest virus, Bebaru virus, Buggy Creek virus, Chikungunya virus, Eastern equine encephalitis virus, Everglades virus, Fort Morgan virus, Getah virus, Highlands J virus, Kyzylagach virus, Mayaro virus, Middelburg virus, Mucambo virus, Ndumu virus Pixuna virus, O'nyong-nyong virus, Ross River virus, Sagiyama virus, Semliki Forest virus, Sindbis virus, Una virus, Venezuelan equine encephalitis virus, Western equine encephalitis virus, or Whataroa virus. In certain embodiments, the synthetic self-replicating RNA is based on a modified Semliki Forest virus, Sindbis virus, or Venezuelan equine encephalitis (VEE) virus, in which the structural genes have been removed. In specific embodiments, the synthetic self-replicating RNA is based on a modified VEE virus, in which the structural genes have been removed. See, e.g., Yoshioka et al. (Cell Stem Cell 13, 246-254, Aug. 1, 2013) and/or Petrakova et al. (J. Virology; vol. 72, no. 12, June 2005, p. 7597-7608) each of which is hereby incorporated by reference herein in their entirety.

The self-replicating RNA comprises sequence encoding a plurality of non-structural replication complex proteins. In specific embodiments, the synthetic self-replicating RNA can encode four non-structural replication complex proteins (i.e., nsP1, nsP2, nsP3, nsP4). The non-structural replication complex proteins can be encoded by a single open reading frame (ORF). In some embodiments, the sequence encoding the non-structural replication complex proteins comprises at least one nucleotide change relative to that of the wild-type virus.

The self-replicating RNA vector further comprises sequence encoding at least one immortalization protein, which are detailed below in section (I)(b).

In general, the self-replicating RNA comprises a 5′ cap, a 5′ untranslated region (UTR) at the 5′ end and a 3′ UTR and a poly A tail at the 3′ end. The self-replicating RNA vector generally comprises a promoter upstream of the sequence encoding the one or more immortalization proteins. The upstream promoter can be a 26S subgenomic promoter.

In some embodiments, the self-replicating RNA can further comprise sequence coding at least one selectable marker. Non-limiting examples of suitable selectable marker include puromycin, geneticin, neomycin, hydromycin B, blastidinin S, and the like.

In other embodiments, the self-replicating RNA can further comprise sequence coding an inhibitor of an interferon response. Examples of suitable interferon response inhibitors include, without limit, vaccinia virus protein E3L, vaccinia virus protein B18R, influenza virus protein NS1, or lymphocytic choriomeningitis virus nucleoprotein.

In still other embodiments, the self-replicating RNA can further comprise sequence coding at least one fluorescent protein. Suitable fluorescent proteins include, without limit, green fluorescent proteins (e.g., GFP, eGFP, GFP-2, tagGFP, turboGFP, Emerald, Azami Green, Monomeric Azami Green, CopGFP, AceGFP, ZsGreenl), yellow fluorescent proteins (e.g., YFP, EYFP, Citrine, Venus, YPet, PhiYFP, ZsYellow1), blue fluorescent proteins (e.g., BFP, EBFP, EBFP2, Azurite, mKalamal, GFPuv, Sapphire, T-sapphire), cyan fluorescent proteins (e.g., ECFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), orange fluorescent proteins (e.g., mOrange, mKO, Kusabira-Orange, Monomeric Kusabira-Orange, mTangerine, tdTomato), or combinations thereof. The fluorescent protein can comprise tandem repeats of one or more fluorescent proteins (e.g., Suntag).

The various protein coding sequences can be separated by internal ribosome entry sequences (IRES) or sequences encoding 2A peptides. Non-limiting examples of suitable 2A peptides include the thosea asigna virus 2A peptide or T2A, foot-and-mouth disease virus 2A peptide or F2A, equine rhinitis A virus 2A peptide or E2A, and porcine teschovirus-1 2A peptide or P2A.

In particular embodiments, the self-replicating RNA can be based on a modified Venezuelan equine encephalitis (VEE) virus and can comprise from 5′ to 3′: a 5′ cap, a 5′ UTR, sequence encoding four non-structural replicases from VEE, a promoter, the sequence encoding immortalization protein(s) (if more than one immortalization protein is encoded, each sequence can be separated by an IRES or 2A peptide sequence), an optional IRES, an optional sequence encoding an E3L protein, an optional IRES, an optional sequence encoding a selectable marker, an alphavirus 3′ UTR, and a poly A tail.

### (b) Immortalization Proteins

The self-replicating RNA vector also comprises sequence encoding at least one immortalization protein. Immortalization proteins confer upon cells the ability to proliferate indefinitely. Non-limiting examples of suitable immortalization proteins include human telomerase (hTert), human papillomavirus type 16 E6 protein (HPV16 E6), human papillomavirus type 16 E7 protein (HPV16 E7), simian vacuolating virus 40 large T antigen (SV40 LT), cMyc-T58A protein, homeobox HoxB8 protein, homeobox HoxA9 protein, homeobox HoxA10 protein, adenovirus E1A protein, adenovirus E1B protein, cyclin-dependent kinase 4 (CDK4), Ras V12 protein, polycomb complex protein Bmi1, hsp70 member 9 (HSPA9), or combination thereof. In particular embodiments, the at least one immortalization protein can be hTert, HPV16 E6-E7, SV40 LT, cMyc-T58A, or combination thereof.

In various embodiments, the at least one immortalization protein can be linked to at least one purification or epitope tag. Non-limiting examples of suitable purification or epitope tags include His, 6×His, Flag, 3×Flag, HA, GST, Myc, SAM, and the like.

In embodiments in which the self-replicating RNA vector encodes more than one immortalization protein, the coding sequences can be separated by internal ribosome entry sequences (IRES) or sequences encoding 2A peptides.

### (c) Specific Embodiments

In one embodiment, the self-replicating RNA vector is based on modified VEE virus and comprises sequence encoding hTert. In another embodiment, the self-replicating RNA vector is based on modified VEE virus and comprises sequence encoding HPV16 E6-E7. In still another embodiment, the self-replicating RNA vector is based on modified VEE virus and comprises sequence encoding SV40 LT. In another embodiment, the self-replicating RNA vector is based on modified VEE virus and comprises sequence encoding hTert and HPV16 E6-E7. In yet another embodiment, the self-replicating RNA vector is based on modified VEE virus and comprises sequence encoding hTert and SV40 LT. In a further embodiment, the self-replicating RNA vector is based on modified VEE virus and comprises sequence encoding hTert and cMyc-T58A.

### (II) Primary Cells Comprising Self-Replicating RNA Vectors

Another aspect of the present disclosure comprises primary cells comprising any one of the self-replicating RNA vectors described above in section (I). That is, the primary cells that have been transfected with one of the self-replicating RNA vectors. Expression of the one or more immortalization proteins in said primary cells enables the cells to become “immortalized” and exhibit extended lifespan and/or undergo additional rounds of cell division, as compared to comparable control cells not transfected with the self-replicating RNA vectors disclosed herein and/or not exposed to immortalization protein(s). Because sequence coding the immortalization proteins is not integrated into the genome of the primary host cells, the immortalization phenotype is reversible by removing the self-replicating RNA vector by dilution and/or via an interferon immune response. Moreover, because sequence coding the immortalization proteins in not integrated into the genome of the host cells, these cells maintain the features and characteristics of the original tissue from which they were isolated.

In some embodiments, the primary cells comprising the self-replicating RNA vectors disclosed herein can further comprise p53 siRNA/shRNA and/or RB siRNA/shRNA. Interference or knockdown of p53 protein and/or RB protein, which block cell cycle progression, may provide additional cell immortalization.

In other embodiments, the primary cells comprising the self-replicating RNA vectors disclosed herein can further comprise vaccinia virus E3L protein, vaccina virus B18R protein, or a combination thereof, which may provide sustained expression of the self-replication RNA vector.

Primary cells are cells isolated directly from human or animal tissue. Non-limiting examples of suitable primary cells include adipocytes, astrocytes, blood cells (e.g., erythroid, lymphoid), chondrocytes, endothelial cells, epithelial cells, fibroblasts, hair cells, hepatocytes, keratinocytes, melanocyte, myocytes, neurons, osteoblasts, skeletal muscle cells, smooth muscle cells, stem cells (e.g., mesenchymal stem cells, hematopoietic stem cells, etc.) or synoviocytes.

In some embodiments, the primary cells may be mammalian. In specific embodiments, the primary cells may be of human origin.

### (III) Plasmid Vectors Encoding Self-Replicating RNA Vectors

A further aspect of the present disclosure provides plasmid vectors encoding the self-replicating RNA described above in section (I). In particular, the plasmid vector comprises sequence encoding the non-structural replication complex proteins from an alphavirus, sequence encoding one or more immortalization proteins, as well as additional viral sequences such as 5′ UTR, subgenomic promoter, and 3′ UTR, optional selectable marker sequence, optional interferon inhibitor sequence, optional IRES, etc.

In general, the plasmid vectors encoding the self-replicating RNA are DNA vectors. The sequence encoding the self-replicating RNA can be operably linked to a promoter sequence that is recognized by a phage RNA polymerase for in vitro RNA synthesis. For example, the promoter sequence can be a T7, T3, or SP6 promoter sequence or a variation of a T7, T3, or SP6 promoter sequence. The promoter sequence can be wild type or it can be modified for more efficient or efficacious expression. The plasmid vector can further comprise at least one transcriptional termination sequence, as well as at least one origin of replication and/or selectable marker sequence (e.g., antibiotic resistance genes) for propagation in bacterial cells. The plasmid vector can be derived from pUC, pBR322, pET, pBluescript, or variants thereof. Additional information about vectors and use thereof can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

Upon in vitro synthesis of the self-replicating RNA, the RNA can be purified, 5′ capped, and polyadenylated using standard procedures or commercially available kits.

### (IV) Methods for Extending Cell Lifespan and Expanding Cell Populations

A further aspect of the present disclosure encompasses methods for extending cell lifespan in a population of primary cells. The methods comprise introducing into the population of primary cells any one of the self-replicating RNA vectors encoding at least one immortalization protein as disclosed herein, wherein upon expression of the at least one immortalization protein, the population of primary cells has an increased lifespan as compared a population of control primary cells not transfected with the self-replicating RNA vector encoding the immortalization protein(s) and/or not exposed to the immortalization protein(s).

The self-replicating RNA vector can be introduced into the population of cells by a variety of transfection means. Suitable transfection methods include nucleofection (or electroporation), calcium phosphate-mediated transfection, cationic polymer transfection (e.g., DEAE-dextran or polyethylenimine), liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids.

In general, the population of cells is maintained under conditions appropriate for cell growth and/or maintenance. Those of skill in the art appreciate that methods for culturing cells are known in the art and can and will vary depending on the type of cells. Routine optimization may be used, in all cases, to determine the best techniques for a particular cell type.

Primary cells have a limited lifespan, which can vary among different types of primary cells and among the same type of cells from different donors. The lifespan (e.g., days, weeks, etc.) can be increased in primary cells transfected with the self-replicating RNA vectors disclosed herein as compared to untreated comparable control cells. In general, the average lifespan in a population of primary cells transfected with the self-replicating RNA vectors disclosed herein can be increased by at least about 20%, at least about 50%, at least about 80%, at least about 1-fold, at least about 1.5-fold, at least about 2-fold, at least about 3-fold, at least about 4-fold, at least about 5-fold, at least about 6-fold, at least about 8-fold, or at least about 10-fold relative to untreated control cells.

Similarly, primary cells have a limited replicative capacity, e.g., they undergo a predetermined, finite number of cell divisions (or cell doublings), which can vary among different types of primary cells and among the same type of cells from different donors. The number of cell divisions can be increased in primary cells transfected with the self-replicating RNA vectors disclosed herein as compared to untreated comparable control cells. In general, the number of cell divisions in a population of primary cells transfected with the self-replicating RNA vectors disclosed herein can be increased by at least about 20%, at least about 50%, at least about 80%, at least about 1-fold, at least about 1.5-fold, at least about 2-fold, at least about 3-fold, at least about 4-fold, at least about 5-fold, at least about 6-fold, at least about 8-fold, or at least about 10-fold relative to untreated control cells.

During replication of the self-replicating RNA vector, there are no DNA intermediates. As such, the replicated RNA cannot recombine with cellular DNA, and there is no risk of integrating immortalization sequences into the genome of the cell. Because there is no genomic integration of oncogenes or immortalization sequences, the integrity of the primary cell is maintained. That is, the primary cells maintain the true characteristics of the original tissue from which they were isolated. Additionally, because there is no genomic integration of oncogenes or immortalization sequences, the stability of the chromosomes is maintained.

The method detailed above can further comprise the step of removing the self-replicating RNA vector from the population of primary cells once the population of primary cells reaches an appropriate cell quantity. The fold expansion or appropriate cell quantity can and will vary depending on the type of cells and the intended use of the expanded cells. The self-replicating RNA vector can be removed from the population of cells over time via dilution. The self-replicating RNA vector also can be removed from the population of cells by allowing (e.g., not suppressing) the strong interferon (IFN) immune response elicited by the RNA vector. For example, the IFN suppressors E3L and/or B18R can be removed from the cells.

Upon removal of the self-replicating RNA vector, the population of expanded primary cells can revert to the original non-immortalized state, e.g., primary cells with a limited lifespan and having the characteristics of the original tissue.

The expanded population of primary cell can be used clinically or therapeutically. Non-limiting therapeutic uses include T cell therapies, mesenchymal stem cell therapies, bone marrow cell therapy, immune cell therapy, and so forth.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “about” when used in relation to a numerical value, x, for example means x ±5%.

The term “expression” with respect to a gene or polynucleotide refers to transcription of the gene or polynucleotide and, as appropriate, translation of an mRNA transcript to a protein or polypeptide. Thus, as will be clear from the context, expression of a protein or polypeptide results from transcription and/or translation of the open reading frame.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “polypeptide” and “protein” are used interchangeably to refer to a polymer of amino acid residues.

As various changes could be made in the above-described cells and methods without departing from the scope of the invention, it is intended that all matter contained in the above description and in the examples given below, shall be interpreted as illustrative and not in a limiting sense.

### EXAMPLES

The following examples illustrate certain aspects of the disclosure.

### Example 1. Construction of Self-Replicating RNA Vectors for Immortalization

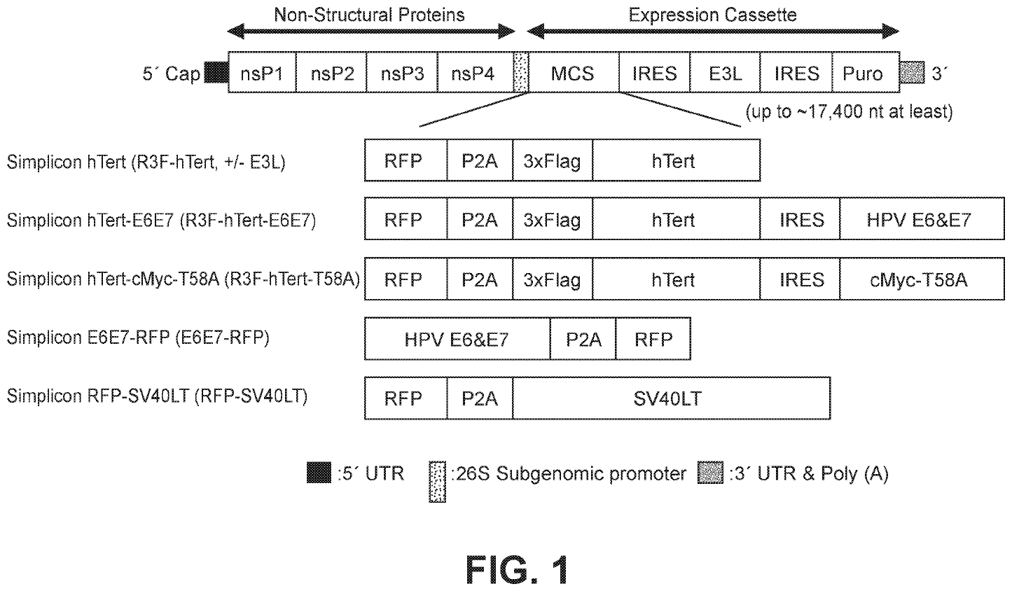

Synthetic, polycistronic, self-replicating RNAs encoding hTert, HPV16-E6E7, cMyc-T58A, SV40LT, or combinations thereof were generated based on a modified Venezuelan equine encephalitis (VEE) virus in which the structural genes have been removed (i.e., Simplicon™ Cloning Vector E3L; MilliporeSigma). Schematics of the various vectors are diagrammed in FIG. 1. hTert was also cloned into a VEE cloning vector that did not encode E3L (E3L suppresses the interferon (IFN) response to self-replication of RNA.)

For RNA synthesis, each vector was linearized and RNA was synthesized and 5′-capped using the RiboMAX Large Scale RNA Production System-T7 (Promega) kit in the presence of CleanCap® Reagent AG (Trilink) for 2 hr at 37° C. In some instances, 5′-capping was performed with enzymatic capping method (e.g., ScriptCap™ Cap1 Capping System). Following purification and precipitation with 2.5 M ammonium acetate, the RNAs were resuspended in the RNA Storage Solution (Ambion) at 1 μg/μl concentration and stored at −80° C.

### Example 2. Immortalization of Human Primary Fibroblasts and Human Mesenchymal Stem Cells

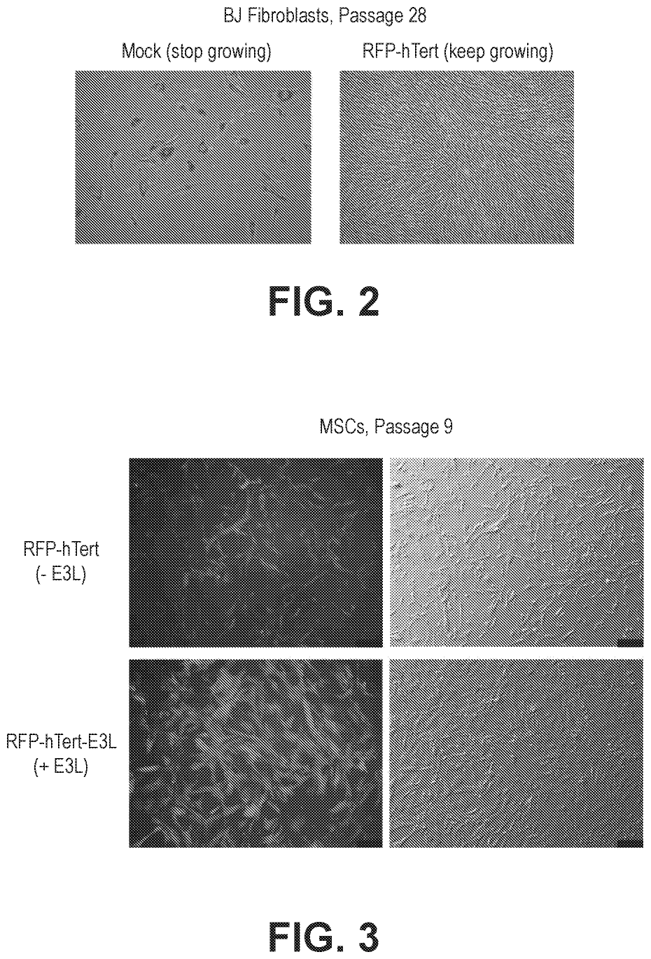

Simplicon-RFP-hTert (no E3L) was transfected into human primary foreskin (BJ) fibroblasts and the cells were continuously cultured in the presence of B18R protein (also suppresses the IFN response) and puromycin. Mock transfected BJ cells stop growing around passage 28, while RFP-hTert transfected BJ cells keep growing after passage 28. Senescence associated beta-galactosidase staining showed the strong staining in mock transfected BJ cells, while there was very weak staining in RFP-hTert expressing BJ cells (FIG. 2).

These results indicated that hTert expression can immortalize human primary fibroblasts.

Human mesenchymal stem cells (MSCs) were transfected with RFP-hTert (no E3L) or RFP-hTert-E3L RNA and continuously cultured in the presence of B18R protein and puromycin. MSCs were passaged for 9 times and RFP expression and proliferation status were captured with the fluorescence microscope (FIG. 3). RFP-hTert-E3L expressing MSCs grew better than the RFP-hTert (no E3L) expressing cells, indicating that E3L expression appears to be required for immortalization of MSCs.

### Example 3. Immortalization of Adult Human Epidermal Keratinocytes Via Combination of hTert and Viral Oncogene

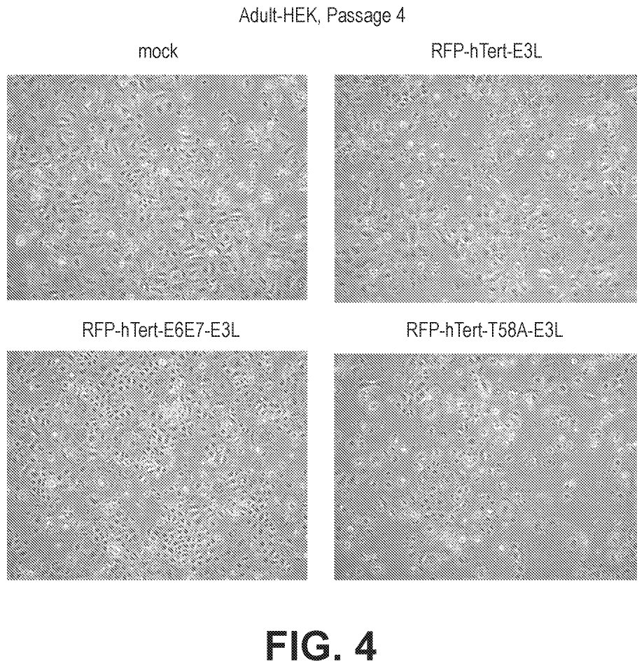

Human epidermal keratinocytes (HEK) cells were transfected with RFP-hTert-E3L, RFP-hTert-E6E7-E3L, or RFP-hTert-T58A-E3L and continuously cultured in the presence of B18R protein and puromycin. Passage 4 cells (4 times passaged cells) were captured and visualized under a microscope. Mock and RFP-hTert-E3L transfected HEK cells showed enlarged, partially differentiated morphology with reduced cell density (indicating slow proliferating), while most of the RFP-hTert-E6E7-E3L transfected HEK cells kept normal morphology and proliferation rate (FIG. 4). The RFP-hTert-T58A-E3L transfected cells were intermediate between the RFP-hTert-E3L and RFP-hTert-E6E7-E3L transfected cells with respect to normal morphology and proliferation. These results indicated that the combination of hTert and HPV16-E6E7 expression may be needed for immortalization of HEK cells.

### Example 4. Recovery of Phenotype after Removal of Simplicon RNA

MSCs were transfected with RFP-hTert-E3L, RFP-hTert-E6E7, or RFP-hTert-T58A RNA, and continuously cultured in the presence of B18R protein and puromycin. MSC cells were passaged four times, then cultured in the absence of B18R protein and puromycin to remove the Simplicon RNA, and then further passaged two times.

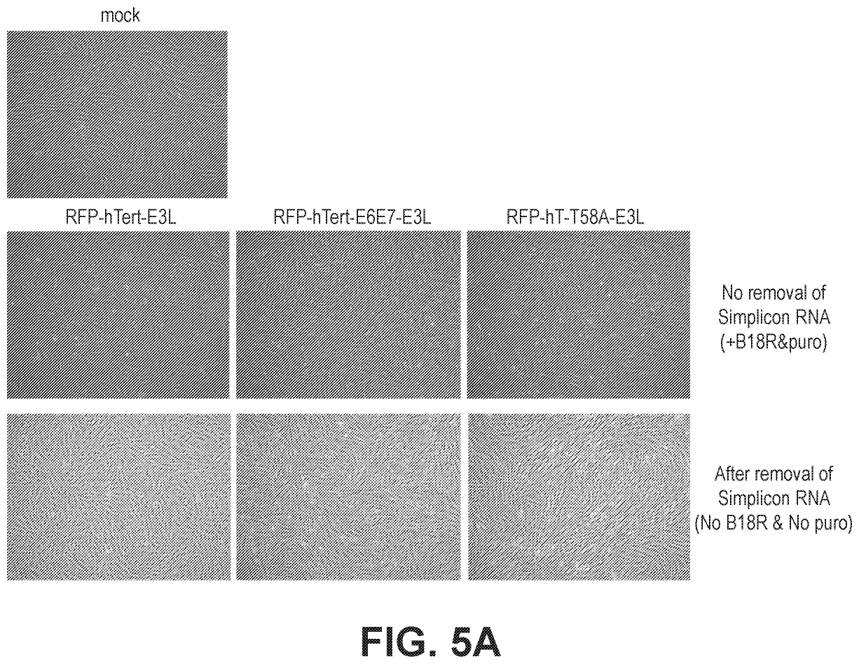

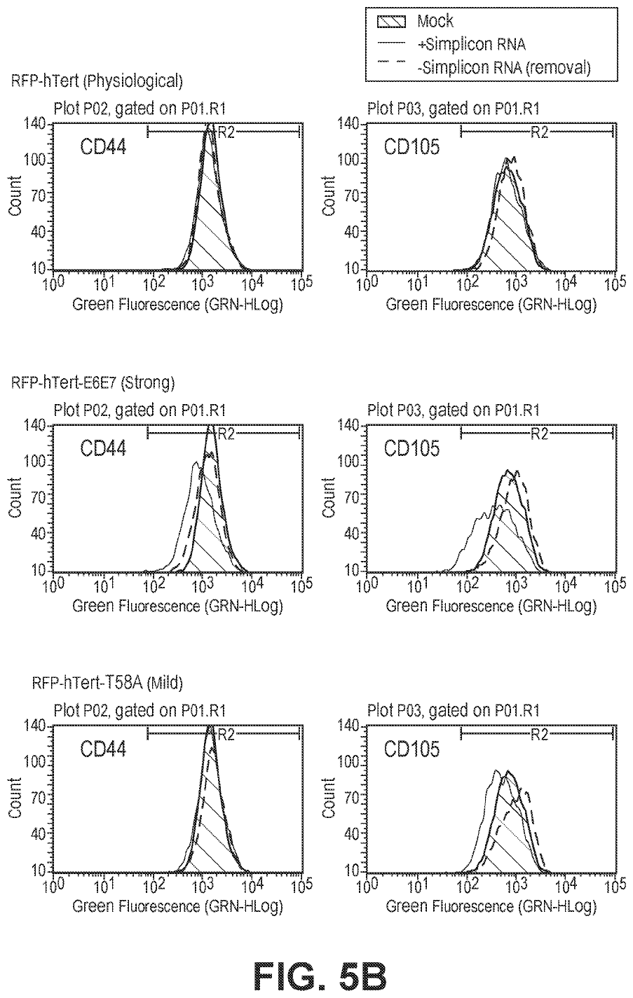

RFP-hTert expressing cells showed normal morphology, while combination of hTert with E6E7 or cMyc-T58A showed somewhat transformed phenotype. These transformed phenotypes recovered after the removal of Simplicon RNA. (FIG. 5A). Cell proliferation rate was also significantly increased by the combination of hTert and E6E7 (data not shown).

The expression of MSC marker genes was examined before and after removal of Simplicon RNA using Alexa Fluor 488 (Green Fluorescence) labelled antibodies and flow cytometry (FIG. 5B). The expression of marker genes such as CD44 and CD105 was not changed in cells immortalized with hTert. Expression of CD44 and CD105 (green line) was decreased in cells immortalized with hTert-E6E7, but expression of these marker genes was restored after removal of Simplicon RNA (blue line). Expression of CD105 marker gene (green line) was decreased in cells immortalized with hTert-T58A, but CD015 expression was restored after removal of Simplicon RNA (blue line).

### Example 5. Immortalization of Adult HEK Cells Via Viral Oncogene Only

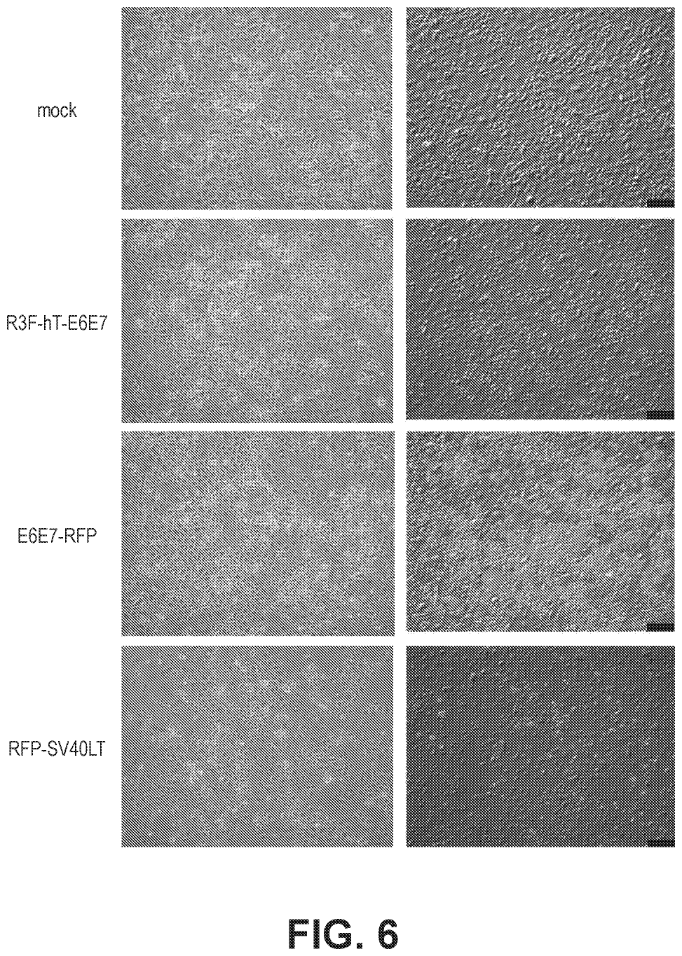

HEK cells were transfected with RFP-hTert-E6E7, E6E7-RFP, or RFP-SV40LT, and then continuously cultured in the presence of B18R protein and puromycin for 7 days. Mock cells showed enlarged and flat phenotype (partially differentiated) and slowed proliferation, while RFP-hTert-E6E7 and E6E7-RFP expressing cells showed normal keratinocytes morphology and keep proliferating. SV40LT transfected cells showed the mixed phenotypes of transformed and normal phenotype (FIG. 6). These data indicate that E6E7 expression may be sufficient for immortalization of HEK cells.

### Example 6. Tuning of Gene Expression Level with the VEE Backbone Mutants

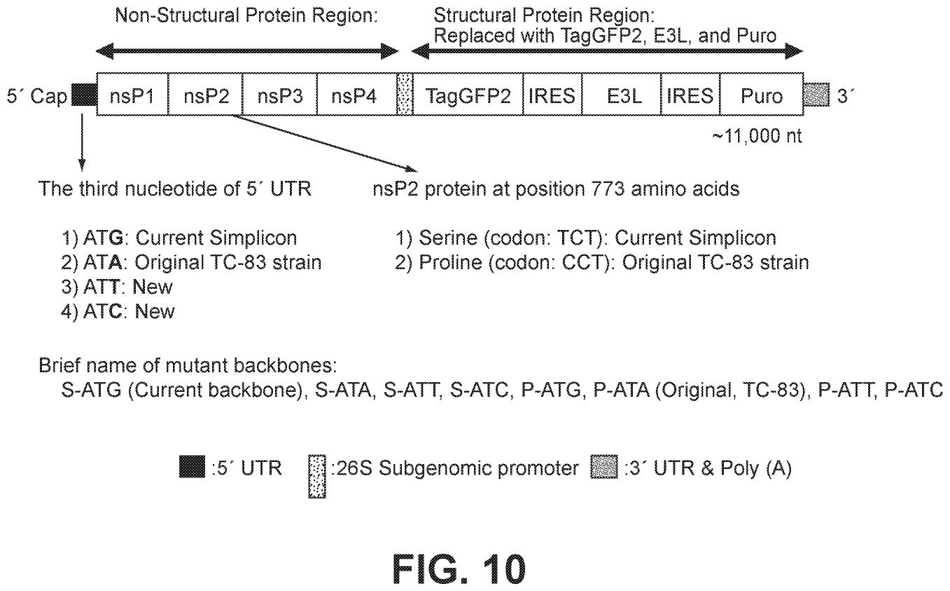

The expression level of immortalization gene(s) affects on efficiency and quality of immortalization. To tune the expression of immortalization gene(s) with a self-replicative RNA, we tested for point mutants of the VEE backbone that has been known to affect on expression and cytopathic effect. First, we tested the third nucleotide of the VEE genome that has been known to affect on cytopathic effect on cells (Journal of Virology, 2005, P7597-7608, doi:10.1128/JVI.79.12.7597-7608.2005). We tested all kinds of nucleotides (A, C, G, T) for GFP expression. Second, we tested the amino acid point mutant at nsP2 protein position 773. The original VEE has “Proline (P)” at this position, while the non-cytopathic mutant has “Serine (5)” at this position. We compared all combinations (Total 8 kinds) of the third nucleotide (A, C, G, T) and nsP2-773 amino acid (P and S) for GFP expression. Briefly, these mutants were named as follows: For “5” or “P” at position nsP2-773 amino acid were labeled as “5” or “P”, respectively, and third nucleotide of “A”, “C”, “G”, and “T” were labeled as ATA, ATC, ATG, and ATT, respectively. All kinds of self-replicative RNAs containing TagGFP2 (S-ATG, S-ATA, S-ATC, S-ATT, P-ATG, P-ATA, P-ATC, and P-ATT) were generated and transfected into HFFs and examined for GFP expression. The location of mutations is summarized in FIG. 10.

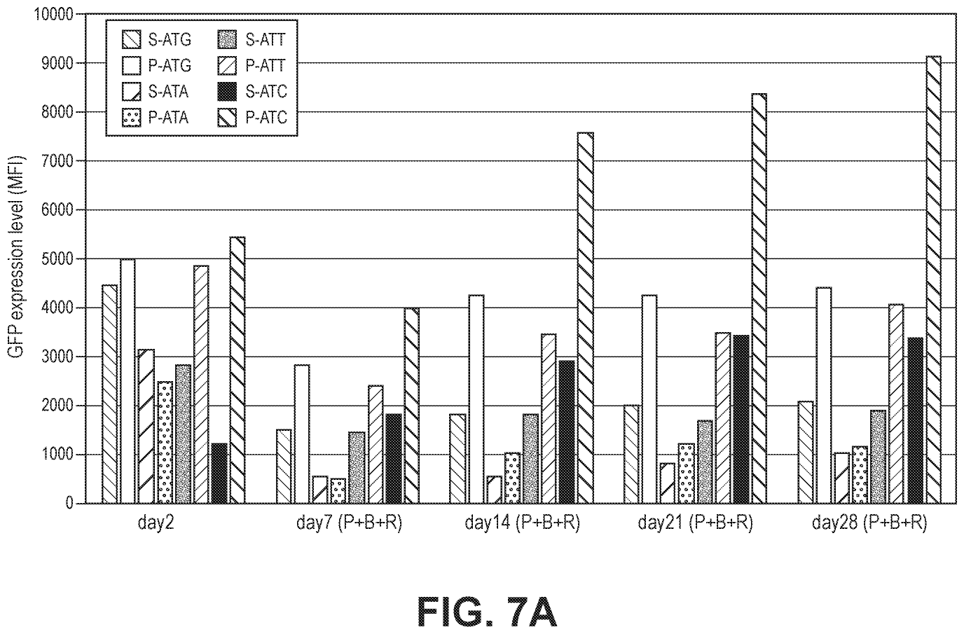

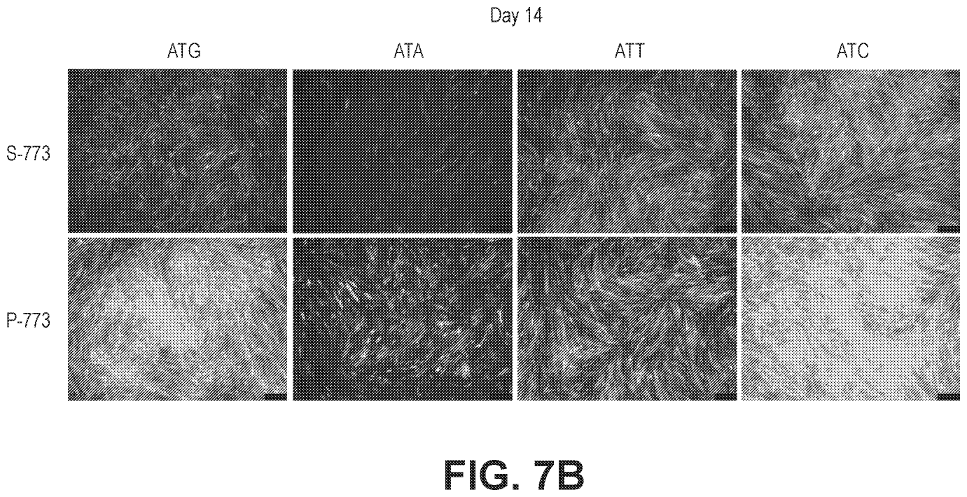

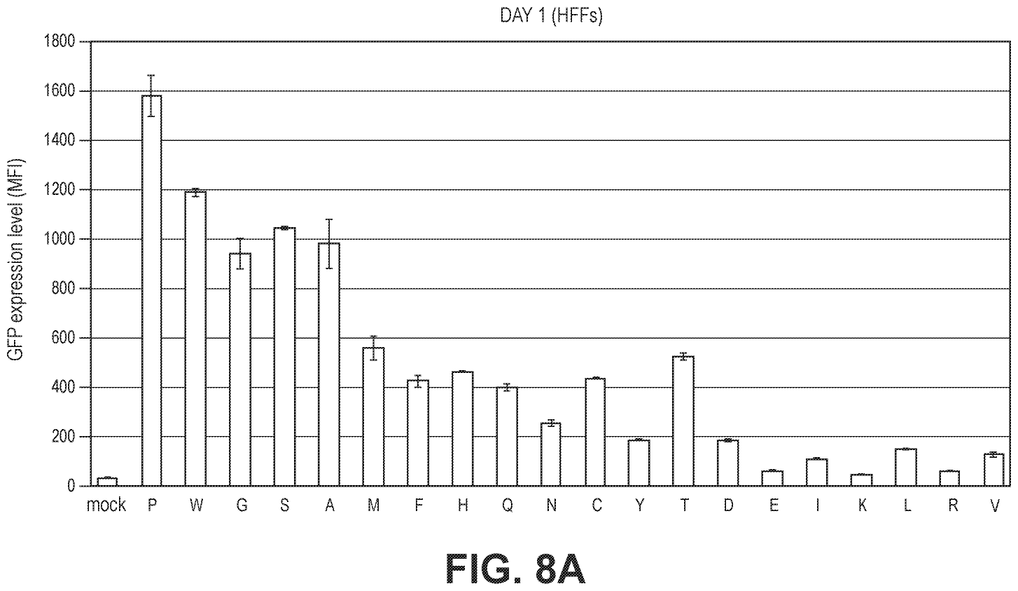

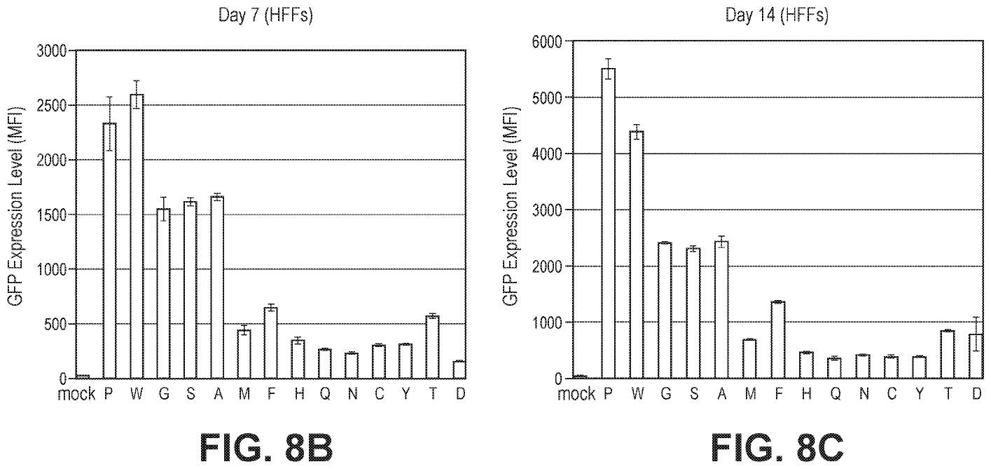

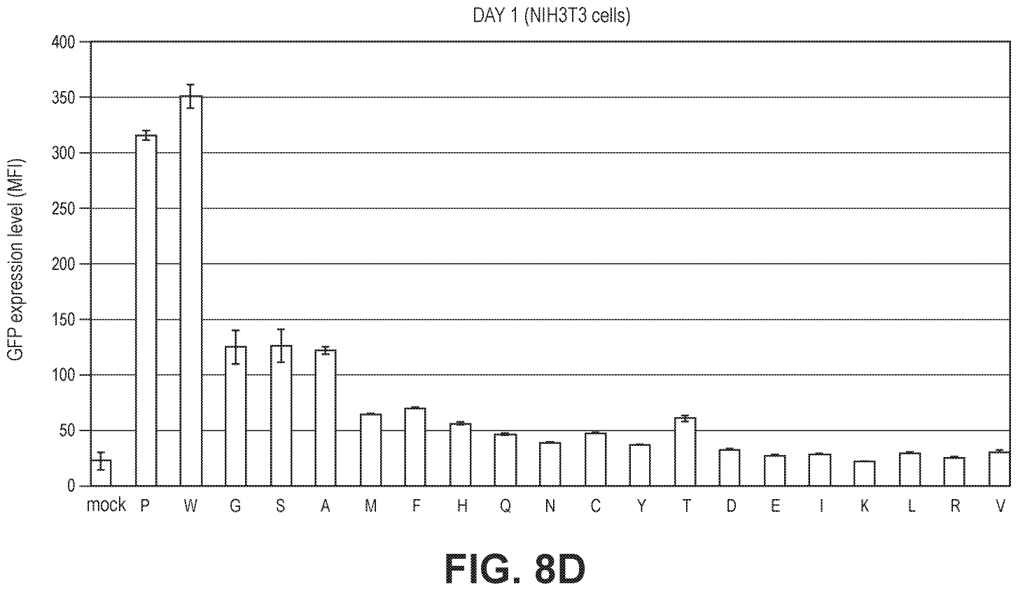

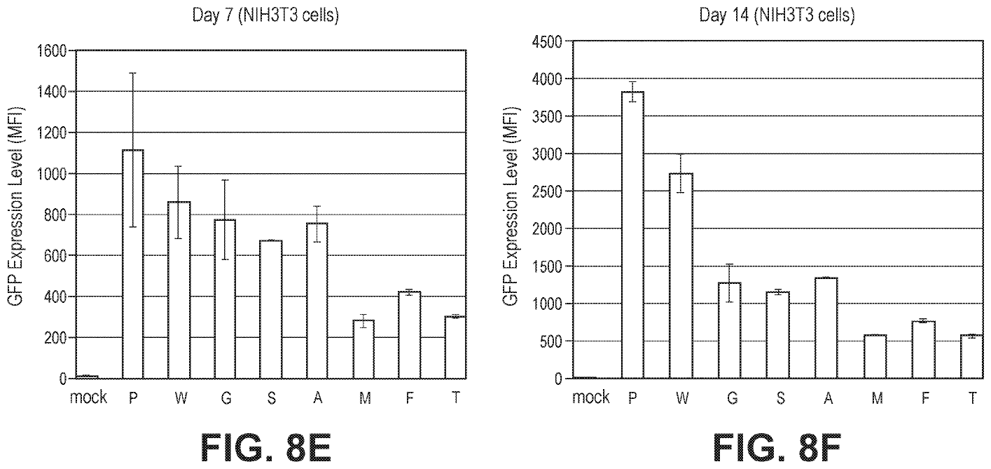

The strong expression was obtained with a P-ATC version in the long-term expression for 4 weeks (FIGS. 7A and 7B). Therefore, we tested more combinations with the third nucleotide “C” (ATC version). Next, we tested all 20 amino acids for the position at nsP2-773 with the ATC version. As shown in FIGS. 8A, 8B, and 8C (Day 1, Day 7, and Day 14, respectively), we obtained the high, middle, low, and no expression levels with the different amino acids at nsP2-773 in HFFs. The “P” and “W” at nsP2-773 position are high, the “G”, “5”, and “A” are middle, and the others are low or no expression. Similar results were obtained when NIH3T3 cells were used (FIGS. 8D, 8E, and 8F). The “M”, “F”, and “T” are expressed low level in both HFFs and NIH3T3 cells. Therefore, we categorized the “M”, “F”, and “T” as a low. In summary, we identified the nsP2-773 mutants “P” and “W” as high, “G”, “5”, “A” as middle, “M”, “F”, “T” as low expression with the third nucleotide “C” (ATC version). These different expression backbones are available for tuning of gene expression.

### Example 7. Immortalization of Adult HEK Cells with the High Expression Backbone (W-ATC)

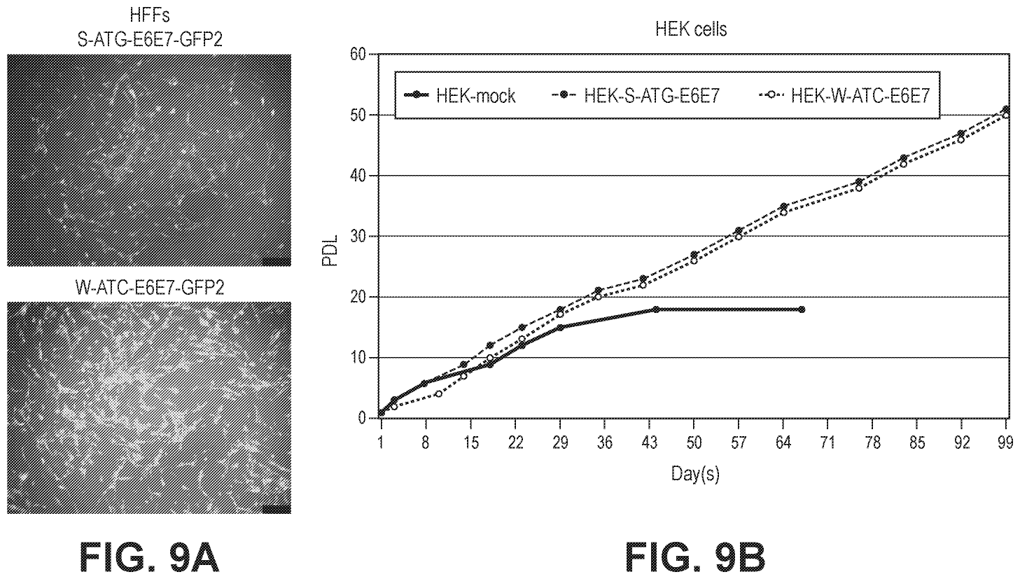

Next, we tested the W-ATC version of VEE backbone for adult human keratinocytes (HEK) immortalization. We generated a self-replicative RNA containing E6E7 oncogene and TagGFP2 with the W-ATC backbone. As shown in FIG. 9A, the W-ATC backbone showed stronger GFP expression than that of the S-ATG backbone in HFFs. Then, we introduced E6E7 oncogene into HEK cells and immortalized HEK cells. As shown in FIG. 9B, HEK-W-E6E7 cells (W-ATC) showed a prolonged life span as well as HEK-S-E6E7 (S-ATG) cells as compared to mock-transfected cells.

1. A self-replicating RNA vector based on an alphavirus in which sequence encoding viral structural proteins is deleted and replaced with sequence encoding at least one immortalization protein.

2. The self-replicating RNA vector of claim 1, wherein the at least one immortalization protein is chosen from human telomerase (hTert), human papillomavirus type 16 E6 protein (HPV16 E6), human papillomavirus type 16 E7 protein (HPV16 E7), simian vacuolating virus 40 large T antigen (SV40 LT), cMyc-T58A protein, homeobox HoxB8 protein, homeobox HoxA9 protein, homeobox HoxA10 protein, adenovirus E1A protein, adenovirus E1B protein, cyclin-dependent kinase 4 (CDK4), Ras V12 protein, polycomb complex protein Bmi1, hsp70 member 9 (HSPA9), or combination thereof.

3. The self-replicating RNA vector of claim 2, wherein the at least one immortalization protein is chosen from hTert, HPV16 E6 and HPV16 E7 (HPV16 E6-E7), SV40 LT, cMyc-T58A, or combination thereof.

4. The self-replicating RNA vector of claim 2, wherein the alphavirus is Aura virus, Babanki virus, Barmah Forest virus, Bebaru virus, Buggy Creek virus, Chikungunya virus, Eastern equine encephalitis virus, Everglades virus, Fort Morgan virus, Getah virus, Highlands J virus, Kyzylagach virus, Mayaro virus, Middelburg virus, Mucambo virus, Ndumu virus Pixuna virus, O'nyong-nyong virus, Ross River virus, Sagiyama virus, Semliki Forest virus, Sindbis virus, Una virus, Venezuelan equine encephalitis (VEE) virus, Western equine encephalitis virus, or Whataroa virus.

5. The self-replicating RNA vector of claim 4, wherein the alphavirus is Semliki Forest virus, Sindbis virus, or VEE.

6. The self-replicating RNA vector of claim 5, wherein the alphavirus is VEE.

7. The self-replicating RNA vector of claim 6, wherein sequence encoding non-structural replication complex proteins of the alphavirus comprises at least one nucleotide change relative to wild-type alphavirus.

8. The self-replicating RNA vector of claim 7, wherein the vector further comprises sequence encoding at least one fluorescent protein.

9. The self-replicating RNA vector of claim 8, wherein the vector further comprises sequence encoding at least one selectable marker.

10. The self-replicating RNA vector of claim 9, wherein the vector further comprises sequence encoding vaccinia virus E3L protein.

11. The self-replicating RNA vector of claim 10, wherein the RNA vector is based on VEE virus and comprises sequence encoding hTert, HPV16 E6-E7, SV40 LT, or cMyc-T58A.

12. The self-replicating RNA vector of claim 10, wherein the RNA vector is based on VEE virus and comprises sequence encoding hTert and HPV16 E6-E7.

13. The self-replicating RNA vector of claim 10, wherein the RNA vector is based on VEE virus and comprises sequence encoding hTert and SV40 LT.

14. The self-replicating RNA vector of claim 10, wherein the RNA vector is based on VEE virus and comprises sequence encoding hTert and cMyc-T58A.

15. A primary cell comprising the self-replicating RNA vector of claim 1.

16. The primary cell of claim 15, further comprising p53 siRNA and/or RB siRNA.

17. The primary cell of claim 15, further comprising vaccinia virus E3L protein, vaccina virus B18R protein, or a combination thereof.

18. The primary cell of claim 17, which is of human origin.

19. The primary cell of claim 18, which is chosen from adipocytes, astrocytes, blood cells, chondrocytes, endothelial cells, epithelial cells, fibroblasts, hair cells, hepatocytes, keratinocytes, melanocyte, myocytes, neurons, osteoblasts, skeletal muscle cells, smooth muscle cells, stem cells, or synoviocytes.

20. A plasmid vector encoding the self-replicating RNA vector as specified in claim 1.

21. The plasmid vector of claim 20, further comprising a T7 or SP6 promoter for in vitro transcription.

22. A method for extending lifespan in a population of primary cells, the method comprising introducing into the population of primary cells the self-replicating RNA vector as specified in claim 1, wherein upon expression of the at least one immortalization protein the population of primary cells has an increased lifespan as compared to a population of control primary cells not transfected with the self-replicating RNA vector and/or not exposed to the at least one immortalization protein.

23. The method of claim 22, wherein the population of primary cells undergoes additional cell divisions as compared to the population of control primary cells.

24. The method of claim 23, further comprising introducing vaccinia virus E3L protein, vaccinia virus B18R protein, or a combination thereof into the population of primary cells.

25. The method of claim 24, further comprising introducing p53 siRNA and/or RB siRNA into the population of primary cells.

26. The method of claim 25, wherein the population of primary cells is of human origin.

27. The method of claim 26, wherein the population of primary cells is chosen from adipocytes, astrocytes, blood cells, chondrocytes, endothelial cells, epithelial cells, fibroblasts, hair cells, hepatocytes, keratinocytes, melanocyte, myocytes, neurons, osteoblasts, skeletal muscle cells, smooth muscle cells, stem cells, or synoviocytes.

28. A method for expanding a population of primary cells, the method comprising:
(a) introducing into the population of primary cells the self-replicating RNA vector as specified in claim 1, wherein upon expression of the at least one immortalization protein the population of primary cells has an increased lifespan as compared to a population of control primary cells not transfected with the self-replicating RNA vector and/or not exposed to the at least one immortalization protein; and
(b) removing the self-replicating RNA vector from the population of primary cells by dilution and/or via an interferon innate immune response once the population of primary cells reaches an appropriate cell quantity.

29. The method of claim 28, wherein at step (a) the population of primary cells undergoes additional cell divisions as compared to the population of control primary cells.

30. The method of claim 29, wherein step (a) further comprises introducing vaccinia virus E3L protein, vaccinia virus B18R protein, or a combination thereof into the population of primary cells.

31. The method of claim 30, wherein step (a) further comprises introducing p53 siRNA and/or RB siRNA into the population of primary cells.

32. The method of claim 31, wherein step (b) comprises removing the self-replicating RNA vector by dilution.

33. The method of claim 32, wherein step (b) comprises removing the self-replicating RNA vector via an interferon immune response.

34. The method of claim 33, wherein the population of primary cells is of human origin.

35. The method of claim 34, wherein the population of primary cells is chosen from adipocytes, astrocytes, blood cells, chondrocytes, endothelial cells, epithelial cells, fibroblasts, hair cells, hepatocytes, keratinocytes, melanocyte, myocytes, neurons, osteoblasts, skeletal muscle cells, smooth muscle cells, stem cells, or synoviocytes.
